(19) United States
(12) Patent Application Publication
(10) Pub. No.: US 2002/0103535 A1
(43) Pub. Date: Aug. 1, 2002

(54) Intraocular lens for double-fold implantation

(76) Inventor: Valdemar Portney, Tustin, CA (US)

Correspondence Address: Howard R. Lambert, 5245 E. Gatewood Lane, Anaheim, CA, 92807, US

(21) Appl. No.: 09/773,100

(22) Filed: Jan. 31, 2001

(51) Int. Cl. A61F 2/16

(52) U.S. Cl. 623/6.18; 623/6.46; 623/6.43

(57) ABSTRACT

A elastically deformable intraocular lens (IOL), adapted for double folding and implanting in the iridocorneal angle or ciliary sulcus of an eye. The IOL includes an optic having a long axis and an orthogonal transverse axis passing through the optical axis and a diameter of between about 5 mm and about 7 mm. First and second fixation members are joined to or at opposite edge regions of the optic. The first fixation member is generally π-shaped, having a base region with a width parallel to the transverse axis no greater than about 3 mm, and includes a pair of similar, spaced apart haptics, proximal ends of which are symmetrically joined to the base region and which diverge outwardly from the long axis at between about 30 and about 45 degrees. The two haptics are constructed to flex in the plane defined by the unflexed haptics during IOL double folding of the IOL and unflex in the same plane after the IOL is implanted in an eye. The second fixation member may be similar to the first fixation member for IOL “four-point” ocular tissue contact or may have only a single haptic for IOL “three-point” ocular tissue contact.

### BACKGROUND OF THE INVENTION

1. Field of the Invention

The present invention relates generally to the field of ophthalmic devices, more particularly to intraocular lenses (IOLs), and still more particularly to foldable or deformable elastic IOLs.

2. Background Discussion

The term “phakic,” refers an eye in which the natural ocular lens is still present, and is in contrast to the term “aphakic” which refers to an eye from which the natural ocular lens has been removed as a result, for example, of cataracts.

Vision in a phakic eye results from light from a viewed object being refracted by the cornea and the natural lens located rearward of the cornea to form an image on the retina at the back of the eye. Such image formation may, for example, be assisted by corrective spectacles, contact lens or corneal reshaping.

The now-common procedure for restoring vision in aphakic eyes (from which diseased or defective natural lenses have been removed) is the ocular implanting of artificial (prosthetic) lenses, called intraocular lenses (IOL), intended to duplicate as closely as possible the optical properties (except for accommodation) of the removed natural lenses.

Considerable attention has more recently been directed toward implanting IOLs in non-cataract phakic eyes to correct vision problems, such as myopia, hypermetropia, presbyopia and astigmatism. Such implanting of corrective IOLs in phakic eyes can eliminate the wearing of spectacles or contact lenses, which are troublesome and, in fact, may limit certain activities and professions, and may be preferred by many individuals as an alternative to permanent surgical cornea reshaping procedures.

My co-pending patent application, Ser. No. 09/690,783 (which is incorporated in its entirety herein by specific reference) discloses an insertion instrument for inserting (implanting) elastically deformable intraocular lenses (IOLs) in eyes. My application Ser. No. 09/690,783 particularly discloses an insertion instrument configured for double folding an elastically deformable IOL for insertion into an eye through a small ocular incision and which provides controlled unfolding of the double folded IOL after its insertion into in a narrow ocular space, for example, the anterior chamber, of an eye, to minimize the possibility of injury to sensitive ocular tissue.

It is thus a principal objective of the present invention to provide an elastically deformable IOL which can be readily double folding (for example, by use of the insertion instrument disclosed in my application Ser. No. 09/690,783) and implanting in the iridocorneal angle of the anterior chamber or ciliary sulcus of the posterior chamber of an eye.

### SUMMARY OF THE INVENTION

In accordance with the present invention, there is provided a double foldable, elastically deformable intraocular lens (IOL) adapted for implanting in the iridocorneal angle of the anterior chamber or ciliary sulcus of the posterior chamber of an eye. The IOL comprises an elastically deformable optic having an optical axis, a long axis and an orthogonal transverse axis both passing through the optical axis, the optic having a diameter between about 5 mm and about 7 mm.

Included in the IOL are first and second fixation members joined to opposite edge regions of the optic for fixating the optic in the eye. The first fixation member is generally pi-shaped having a base region with a length parallel to the optic transverse axis not greater than about 3 mm, and includes a pair of flexible haptics symmetrically positioned relative to the optic long axis and diverging outwardly from the optic. Each of the haptics has a proximal end joined to the base region and a free distal end.

Preferably each one of the pair of flexible haptics is substantially continuously curved to provide a spring-like flexibility and is constructed to be more flexible in a region adjacent the proximal end than in a region adjacent the distal end. Moreover, it is preferred that the pair of flexible haptics be constructed from a material that is stiffer than the optic and that the distal end of each one of the pair of flexible haptics be shaped to provide a ocular tissue line contact upon the implanting of the IOL in the eye.

In accordance with a preferred embodiment, each one of the pair of flexible haptics diverges from the optic long axis at an angle, α, of between about 30 degrees and about 45 degrees.

The second fixation member may include a pair of diverging haptics and be a mirror image of the first fixation member so as to provide, in combination, a “four-point” line contact with ocular tissue or may comprise a single flexible haptic extending from the optic generally along the long axis so as to provide, in combination, a “three-point” line contact with ocular tissue.

More specifically, the double foldable, elastically deformable intraocular lens (IOL) may comprise an elastically deformable optic having a diameter of between about 5 mm and about 7 mm and having an optical axis, a long axis and an orthogonal transverse axis both of which pass through the optical axis. The IOL further comprises first and second flexible fixation members joined to opposite edge regions of the optic for fixating the optic in the eye.

The first fixation member is generally pi-shaped and includes a base region joined to the optic and having a length parallel to the optic transverse axis of no more than about 3 mm and a radial width of about 0.25 mm, and includes a pair of similar, flexible haptics, proximal ends of which are joined to the base region in a spaced apart relationship. The two haptics are symmetrically positioned relative to the optic long axis and diverge outwardly from the long axis at an angle between about 30 degrees and about 45 degrees. The flexible haptics are preferably constructed from a material that is stiffer than the material from which the optic is constructed.

The farthest apart outside edges of the pair of flexible haptics are separated by no more than about 7 mm. Each one of the flexible haptics has a substantially continuously curved shape and is more flexible in a proximal end region than in a distal end region, the distal end region being shaped to provide a linear ocular tissue contact upon the implanting of the IOL in said eye. Distal ends of the pair of haptics lie on a diameter of between about 11.5 mm and about 13.5 mm that defines the overall diameter of the IOL.

The second fixation member may be constructed similar to the first fixation member to thereby provide, in combination, a “four-point” linear ocular tissue contact; alternatively, the second fixation member may a single flexible haptic fixed to a base region to thereby provide, in combination, a “three-point” linear ocular tissue contact.

The two haptics comprising the first fixation member and, if applicably, the two haptics comprising the second fixation member, are constructed so that the haptics forming each fixation member flex inwardly toward one another upon double folding of the IOL, and unflex outwardly to their initial unflexed condition after implanting in an eye. Both the inward flexing and the outward unflexing of haptics are confined by the base region (or regions) to occur in a general plane defined by the initially unflexed haptics.

### BRIEF DESCRIPTION OF THE DRAWINGS

The present invention can be more readily understood by a consideration of the following detailed description when taken in conjunction with the accompanying drawings, in which:

FIG. 1 is a plan view of an elastically deformable intraocular lens (IOL) adapted for double folding and implanting in the iridocorneal angle of the anterior chamber or ciliary sulcus of the posterior chamber of a human eye, showing an optic and two fixation members fixed to opposite side edges of the optic, both fixation members being shown in solid lines as comprising a mirror-image pair of fixation elements (haptics) to provide four point ocular tissue contact, there being shown in broken lines a single fixation element comprising one of the two fixation members so that a three-point ocular tissue contact is provided;

FIG. 2 is a side view of the IOL depicted in FIG. 1, showing, by way of illustrative example, the IOL implanted in the iridocorneal angle of the anterior chamber of a representative human eye, and also depicting a previously implanted IOL in the posterior chamber of the eye;

FIG. 3 is a plan view of a variation elastically deformable intraocular lens (IOL) similar to the IOL shown in FIG. 1, showing tissue contacting distal end regions of fixation member haptics reversed relative to the tissue contacting distal end regions of the fixation haptics shown in FIG. 1;

FIG. 4 is a four step sequence showing the double folding of the representative IOL of FIG. 1 in the IOL double folding instrument disclosed in my co-pending application Ser. No. 09/690,783 and the partial ejecting of the double folded IOL from the instrument insertion tip: FIG. 4A showing the IOL moved axially in the double folding instrument with side edge regions of the leading pair of the IOL fixation haptics about to engage a converging inner surface of a double folding member; FIG. 4B showing the IOL moved further axially in the double folding instrument with the leading pair of IOL haptics now deflected toward each other by engagement with the double folding member converging inner surface and with the leading edge of the IOL optic just entering the double folding member; FIG. 4C showing the IOL downstream of the double folding member and at the IOL insertion tip of the instrument, showing both the leading and trailing pairs of fixation haptics deflected toward, and partially overlapping, each other and showing the IOL optic in its fully double folded condition; and FIG. 4D showing the leading pair of IOL fixation haptics just outside the open end of the instrument insertion tip and elastically returned to their initial spread apart condition and showing major regions of the IOL optic unfolding outside the instrument insertion tip; and

FIG. 5 is a transverse cross sectional view looking along line 5-5 of FIG. 4C, showing the oval cross sectional shape of the insertion tube disposable insertion tip and showing the double folded IOL optic inside the insertion tip.

In the various FIGS. the same elements and features are given the same reference numbers while corresponding features and elements are given the same reference number followed by the letter “a”.

### DESCRIPTION OF THE PREFERRED EMBODIMENT

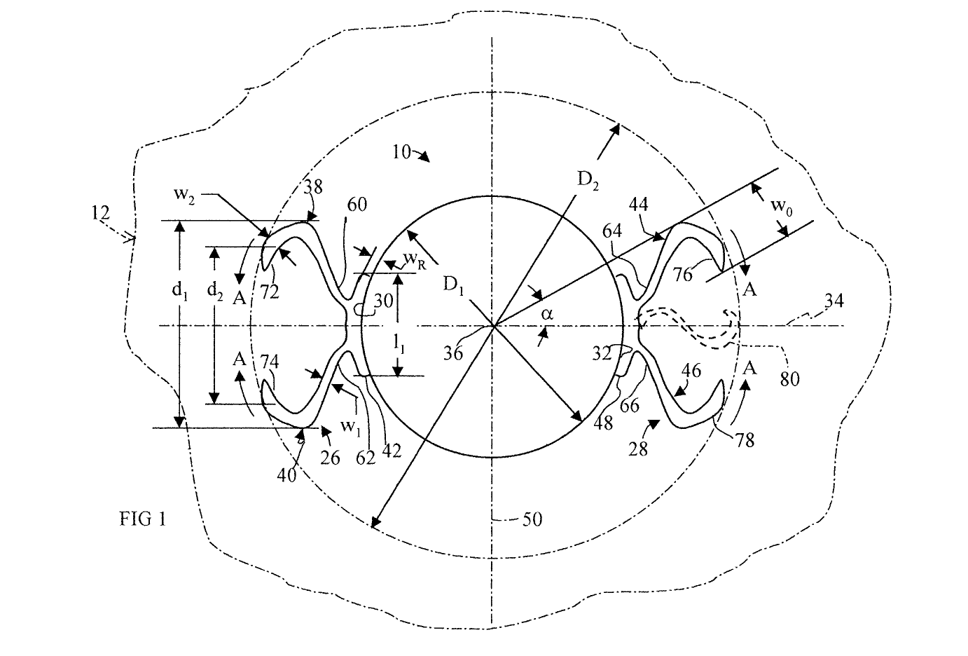

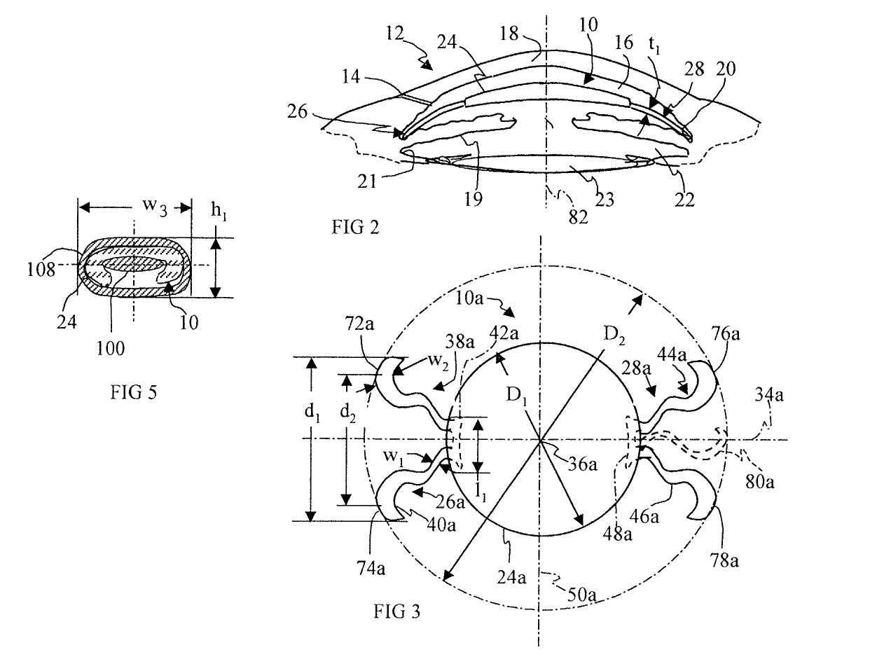

There is shown in FIG. 1, an elastically deformable intraocular lens (IOL) 10 in accordance with a preferred embodiment of the present invention. As described below, IOL 10 is configured for being double folded, for example, in the double folding instrument disclosed in my co-pending application Ser. No. 09/690,783, and implanted in an eye 12 through a small ocular incision 14 (FIG. 2) for unfolding in narrow anterior chamber 16 (between cornea 18 and iris 19) for placement at iridocorneal angle 20 to correct vision in the eye.

Alternatively (not shown) IOL 10 may be implanted in ciliary sulcus 21 of eye 12 in narrow posterior chamber 22 between iris 19 and a previously implanted IOL 23 to correct optical deficiencies (such as diopter) of the already-implanted IOL and improve vision in an aphakic eye.

Shown in FIG. 1 comprising IOL 10 is an optic 24, which, as shown by way of illustrative example, circular in outline having an outer diameter, D₁, that may be between about 5 mm and about 7 mm. As depicted in FIG. 2 by way of further example, IOL 10 may be slightly vaulted and optic 24 may be of a concave-convex configuration.

Also comprising IOL 10 are respective first and second fixation means or members 26 and 28 that are fixed or joined to respective opposite edge regions 30 and 32 of optic 24. Distal ends of fixation members 26 and 28, in their un-flexed condition, lie on or define a maximum IOL diameter, D₂, which may, for example, be between about 11.5 mm and about 13.5 mm.

As further shown in FIG. 1, first and second fixation members 26 and 28, which are generally pi-shaped (π-shaped), are symmetrical about a long axis 34 (axis of symmetry) that passes through a optical center 36 of optic 24 and through the center of the two fixation members.

First fixation member 26 comprises respective first and second elastically flexible, substantially continuously curved fixation elements or haptics 38 and 40 that diverge outwardly from an interconnecting base region 42 fixed or joined to or at optic side edge region 30. Second fixation member 28 is, as shown in FIG. 1 in solid lines, formed like first fixation member 26 and comprises respective first and second elastically flexible, substantially continuously curved fixation elements or haptics 44 and 46 that diverge outwardly from an interconnecting base region 48 fixed or joined to or at optic side edge region 32.

Although each of base regions 42 and 48 are shown in FIG. 1 as being continuous, as is preferred, the base regions may alternatively comprise two (or even more) abutting sections (not shown).

An angle, α, to outside edges of haptics 38, 40, 44 and 46 (shown in FIG. 1 only for representative haptic 44) is preferably between about 30 degrees and about 45 degrees relative to optic long axis 34. First and second fixation members 26 and 28 are shown in FIG. 1 symmetrical to one another about a transverse axis 50 that also extends through optic center 36 and is orthogonal to long axis 34.

First and second fixation member haptics 38, 40, 44 and 46 provide what is conventionally referred to as a “four-point” contact with receiving ocular tissue (for example, iridocorneal angle 20 or ciliary sulcus 21) upon implanting of IOL 10 in eye 12, each such haptic being shaped and constructed to elastically flex in the direction of arrow “A” (FIG. 1) toward long axis 34 when IOL 10 is double folded, as more particularly described below.

Each of respective base regions 42 and 48 of first and second fixation members 26 and 28 has a length, l₁, parallel to transverse axis 50 that may be about 2.5 mm and preferably is no more than about 3 mm. and may have a width, W_{R}, in a radial direction of about 0.25 mm (shown in FIG. 1 only for representative fixation member 26). Proximal end regions 60 and 62 of first fixation member haptics 38 and 40 and respective proximal end regions 64 and 66 of second fixation member haptics 44 and 46 are spaced apart on respective base regions 42 and 48, within the associated base region lengths, l₁,

The most divergent (widest spaced apart) outer edges of haptics 38 and 48 and of haptics 44 and 46 are spaced apart a distance, d₁, (shown in FIG. 1 for representative haptics 38 and 40) that is preferably about 6 mm.

Overall width, W₀, of haptics 38, 40, 44 and 46 (shown for representative haptic 44 in FIG. 1) is preferably about 2.5 mm. Respective proximal end regions 60, 62, 64 and 66 of haptics 38, 40, 44 and 46 have widths, W₁ (shown for representative haptic 38) of about 0.2 mm, while respective distal (free) end regions or feet 72, 74, 76 and 78 of haptics 38, 40, 44 and 46 have increased widths, W₂ (also shown for representative haptic 38), that are preferably about 0.3 mm. Haptics 38,40, 44 and 48 preferably have a uniform thickness, t₁, that is about 0.25 mm (FIG. 2).

Because haptics 38, 40, 44 and 46 increase in width from proximal end regions 60, 62, 64 and 66 to distal end regions 72, 74, 76 and 78, the haptic proximal end regions are more elastically flexible than the haptic distal end regions and thus the haptics elastically flex (bend) principally about their associated proximal end regions more during the double folding procedure described below.

As further shown in FIG. 1, respective distal end regions 72 and 74 of haptics 38 and 40 are curved inwardly toward long axis 34 and are generally tangent to diameter, D₂, thereby providing spaced apart receiving tissue contact lines when IOL 10 is implanted in eye 12. Central tissue contact points of distal end regions 72 and 74 of fixation member 26 are separated by a distance, d₂, that is preferably about 5 mm. This same distance, d₂, separates central tissue contact points of distal end regions 76 and 78 of fixation member 28.

In combination, fixation members 26 and 28 provide what is conventionally referred to (in the IOL industry) as a “four-point” fixation or ocular tissue contact upon implanting of IOL 12 in eye 12. However, haptic curved distal end regions 72, 74, 76 and 78 actually provide small line rather than point contact with ocular tissue that prevents injury to, while avoiding interaction with, the active trabecular network of iridocorneal angle 20 or the active ciliary body of ciliary sulcus 21 (depending upon the ocular implanting location).

Fixation member base region 42 importantly constrains respective haptics 38 and 40 to flex inwardly toward long axis 34 in a plane generally defined by those two initially unflexed haptics. In a like manner, fixation member base region 48 constrains respective haptics 44 and 46 to flex inwardly toward long axis 34 in the plane substantially defined by those two initially unflexed haptics. This same constraining function is provided by base regions 42 and 48 when double folded IOL is released in anterior chamber 16 or posterior chamber 22 and haptic pairs 38, 40 and 44, 46 unflex back to their initial unflexed condition. (It should be noted that in the case of un-vaulted or only slightly vaulted IOLs, the above-described inward flexing and outward unflexing of haptic pairs 38, 40 and 44, 46 will be in a generally common plane that is generally in the plane of optic before its double folding.)

It is important that the above-described construction of fixation members 26 and 28 constrains associated haptics 38, 40 and 44, 46 (after having been flexed inwardly during the IOL double folding procedure) to unflex in two-dimensional space as double folded IOL 10 unfolds in the narrow space of an anterior chamber 16 or posterior chamber 22 of eye 12. Such constrained, two-dimensional (planar) unflexing of haptic pairs 38, 40 and 44,46 as double folded IOL 10 is released in an eye substantially reduces the risk that the released haptics will hit and injure sensitive ocular tissue associated with the narrow anterior and posterior chambers of the eye.

This above-described constrained two-dimensional unflexing of haptics 38-46 of unfolding IOL 10 is in significant contrast to the unconstrained three-dimensional haptic unflexing of known elastically deformed IOLs which can cause injury to sensitive ocular tissue upon unfolding of the IOL in narrow regions of an eye.

Fixation member base regions 42 and 48 also function to stabilize optic 24 after IOL 10 is implanted in an eye and inhibit twisting of the optic about long axis 34 which could cause vision distortions. Such stabilization is particularly important for very thin optics 24.

There has been described above the construction of IOL 10 with fixation members 26 and 28 considered together having four haptics 38, 40, 44 and 46 that provide “four-point” contact or fixation in ocular tissue upon implanting of the IOL in an eye. The present inventor however considers it within the scope of his invention to eliminate one of the haptics in one of fixation members 26 and 28 to thereby provide a “three-point” contact or fixation in ocular tissue contact upon implanting the resulting IOL in an eye.

Such a “three-point” contact or fixation configuration is also depicted in FIG. 1, in which a single haptic 80, corresponding to described haptic 46 and shown in broken lines along long axis 34, is substituted for haptics 44 and 46 of fixation member 28. All other features and dimensions of the resulting “three-point” fixation IOL would, however, be the same as described for IOL 10.

It should be further noted that in FIG. 1, IOL 10 is depicted as a one-piece IOL, which may be made from an elastomeric material, such as silicone or acrylic, with fixation members 26 and 28 formed integrally with optic 24. Although, fixation members 26 and 28 may still be composed of a stiffer elastically deformable material than optic 24 in accordance with known IOL construction practices.

### Variation IOL of FIG. 3

FIG. 3 depicts a variation IOL 10a that corresponds, except as specifically described, to above-described IOL 10 (original reference numbers followed by an “a” being used as appropriate for IOL 10a). As shown, IOL 10a comprises an optic 24a (of diameter, D₁,) and respective first and second fixation members 26a and 28a defining overall diameter, D₂. (IOL diameters D₁ and D₂ of IOL 10a being preferably as disclosed above for IOL 10.)

Variation IOL 10a is shown in FIG. 3, by way of example only, as a three-piece IOL with optic 24a molded over and around respective base regions 42a and 48a of fixation members 26a and 28a. As described above for base regions 42 and 48 of IOL 10, base regions 42a and 48a of IOL 10a may be continuous, as shown in FIG. 3, or may be formed in two or more abutting sections. Optic 24a may be constructed of an elastically flexible silicone or acrylic material. Fixation members 26a and 28a may, for example, be constructed from polymethyl methacrylate (PMMA) to be elastically deformable due to their size and shape.

Fixation member 26a comprises, in addition to base region 42a, first and second haptics 38a and 40a having respective distal end regions or feet 72a and 74a. Fixation member 26a corresponds to above-described fixation member 26, with haptics 38a and 40a being similar to above-describe haptics 38 and 40 (FIG. 1) except for being reversed 180 degrees so that distal end regions 72a and 74a are directed away from one another and away from optic long axis 34a.

Similarly, fixation member 28a comprises, in addition to base region 48a, first and second haptics 44a and 46a having respective distal end regions or feet 76a and 78a. Fixation member 28a corresponds to above-described fixation member 28, with haptics 44a and 46a being similar to above-describe haptics 44 and 46 (FIG. 1) except for being reversed 180 degrees so that distal end regions (feet) 76a and 78a are directed away from one another and away from optic long axis 34a.

Lengths, l₁, and widths, W_{R}, of fixation member base regions 42a and 48a are as described above for corresponding fixation member base regions 42 and 48. Distance, d₁, to outermost points of haptics 38a and 40a, as well as haptics 44a and 46a, are as described above, as is distance, d₂, between central contact regions of haptic distal end region pairs 72a, 74a and 76a, 78a.

As described above relative to IOL 10, the two haptics 44a and 46a of fixation member 28a may be replaced by a single haptic 80a shown in broken lines.

### IOL Double Folding and Implanting Operation

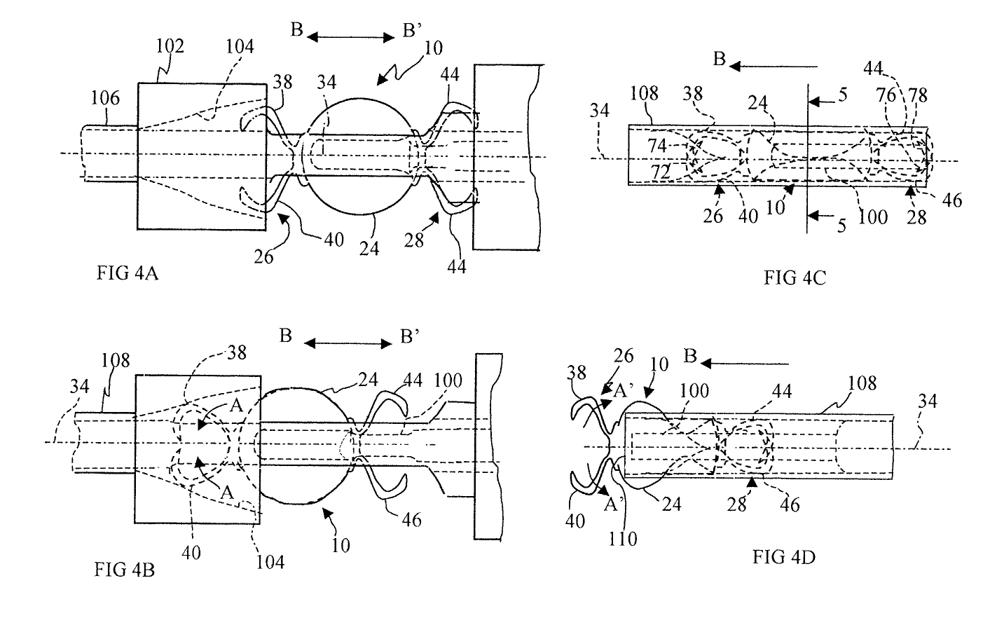

FIG. 4 depicts, in a sequence of four steps, the double folding and discharging of representative IOL 10, using the double folding instrument disclosed in my co-pending application Ser. No. 09/690,783.

FIG. 4A shows IOL 10 pushed by a pushing member 100, in the axial direction of Arrow B, into an IOL double folding member 102 having a converging inner surface 104 and mounted on an insertion tube 106. As shown, IOL leading fixation member 26 has just entered double folding member 102, with leading haptics 38 and 40 about to engage converging inner surface 104. (Alternatively, IOL double folding member 102 may be moved in the opposite axial direction of Arrow B′ over IOL 10.)

In FIG. 4B, IOL 10 is shown pushed by member 100 further in the direction of Arrow B, with leading haptics 38 and 40 now flexed inwardly (direction of Arrows A) toward one another and optic long axis 34 in response to being slid along double folding member converging inner surface 104. IOL optic 24 is shown just entering double folding member 102, but not yet engaging converging inner surface 102. (The same result can be achieved by alternatively moving double folding member 102 over IOL 10 in the axial direction of Arrow B′.)

FIG. 4C shows IOL 10 completely double folded and pushed by member 100 into a disposable tip 108 at the discharge end of tube 106. At this point, IOL optic 24 has been doubled folded by being pushed in the axial direction of Arrow B through double folding member 102 and leading haptics 38 and 40 and trailing haptic 44 and 46 have been flexed together with haptic distal end region pairs 72, 74 and 76, 78 overlapping one another at long axis 34.

IOL 10 is shown in FIG. 4D further pushed by member 100 in the direction of Arrow B so that the IOL is being discharged from distal end 110 of disposable tip 108. As shown, released lead haptics 38 and 40 have unflexed (direction of Arrows A′) back to their original, unflexed condition (depicted in FIG. 1). IOL optic 24 is shown partially unfolded with trailing haptics 44 and 46 still maintained inwardly flexed in tip 108.

FIG. 5 shows IOL optic 24 double folded into an inverted C-shape inside insertion tip 108 that has an outside width, W₃, that is preferably no greater than about 3.5 mm and that has an outside height, h₁, that is preferably no greater than about 1.3 mm.

It is, of course, to be understood that FIGS. 4 and 5 apply as well to above-described IOL 10a and to both above-described “three-point” contact versions of IOLs 10 and 10a.

Although there has been described above an elastically deformable intraocular lens and variations thereof adapted for double folding and implanting into a narrow space in a patients eye, especially in the anterior chamber in the iridocorneal angle or in the posterior chamber in the ciliary sulcus, in accordance with the present invention for purposes of illustrating the manner in which the present invention maybe used to advantage, it is to be understood that the invention is not limited thereto. Consequently, any and all variations and equivalent arrangements that may occur to those skilled in the applicable art are to be considered to be within the scope and spirit of the invention as set forth in the claims, which are appended hereto as part of this application.

What is claimed is:

1. A double foldable, elastically deformable intraocular lens (IOL) adapted for implanting in the iridocorneal angle of the anterior chamber or ciliary sulcus of the posterior chamber of an eye, said IOL comprising:
a. an elastically deformable optic having an optical axis, a long axis passing through said optical axis and a transverse axis passing through said optical axis orthogonal to said long axis; and
b. first and second flexible fixation members joined to said optic for fixating said optic in said eye, said first fixation member including a base region and a pair of flexible haptics having is proximal ends joined to said base region symmetrically about said long axis and diverging outwardly from said long axis, said base region being joined to or at an edge region of said optic and having a length parallel to said transverse axis no greater than about 3 mm.

2. The double foldable, elastically deformable intraocular lens (IOL) as claimed in claim 1, wherein each one of said pair of flexible haptics has a substantially continuously curved shape to provide a spring-like flexibility.

3. The double foldable, elastically deformable intraocular lens (IOL) as claimed in claim 1, wherein each one of said pair of flexible haptics is constructed so as to be more flexible in a region adjacent said proximal end than in a region adjacent said distal end.

4. The double foldable, elastically deformable intraocular lens (IOL) as claimed in claim 1, wherein a distal end of each one of said pair of flexible haptics is shaped to provide an ocular tissue line contact area upon the implanting of said IOL in said eye.

5. The double foldable, elastically deformable intraocular lens (IOL) as claimed in claim 1, wherein said pair of flexible haptics are constructed from a material that is stiffer than said optic.

6. The double foldable, elastically deformable intraocular lens (IOL) as claimed in claim 1, wherein each one of said pair of flexible haptics diverges at an angle, α, relative to said optic long axis that is between about 30 degrees and about 45 degrees.

7. The double foldable, elastically deformable intraocular lens (IOL) as claimed in claim 1, wherein each one of said pair of flexible haptics is a mirror image of the other one of said pair of flexible haptics.

8. The double foldable, elastically deformable intraocular lens (IOL) as claimed in claim 1, wherein both of said second fixation member is constructed similar to said first fixation member.

9. The double foldable, elastically deformable intraocular lens (IOL) as claimed in claim 1, wherein said second fixation member comprises a single curved flexible haptic, said single flexible haptic extending from said optic generally along said optic long axis.

10. A double foldable, elastically deformable intraocular lens (IOL) adapted for implanting in the iridocorneal angle of the anterior chamber or ciliary sulcus of the posterior chamber of an eye, said IOL comprising:
a. an elastically deformable optic having an optical axis, a long axis passing through said optical axis and a transverse axis passing through said optical axis orthogonal to said long axis, said optic having a diameter between about 5 and about 7 mm; and
b. first and second flexible fixation members joined to or at opposite edge regions of said optic for fixating said optic in said eye, said first fixation member being generally π-shaped and including a base region joined to the optic and a pair of flexible haptics, proximal ends of said haptics being joined to said base region in a mutually spaced apart relationship symmetrically positioned relative to said long axis and diverging outwardly from said long axis at an angle between about 30 degrees and about 45 degrees, said base region having a length parallel to said transverse axis no greater than about 3 mm.

11. The double foldable, elastically deformable intraocular lens (IOL) as claimed in claim 10, wherein farthest apart outside edges of said pair of flexible haptics are separated by no more than about 7 mm.

12. The double foldable, elastically deformable intraocular lens (IOL) as claimed in claim 10, wherein each one of said pair of flexible haptics has a substantially continuously curved shape and is more flexible in a proximal end region than in a distal end region and wherein said distal end region is shaped to provide an ocular tissue line contact upon the implanting of said IOL in said eye.

13. The double foldable, elastically deformable intraocular lens (IOL) as claimed in claim 10, wherein said pair of flexible haptics are constructed from a material that is stiffer than said optic.

14. The double foldable, elastically deformable intraocular lens (IOL) as claimed in claim 10, wherein said second fixation member is constructed similar to said first fixation member so as to provide a four-point contact IOL.

15. The double foldable, elastically deformable intraocular lens (IOL) as claimed in claim 10, wherein said second fixation members comprises at a single flexible haptic so as to provide a three-point contact IOL.

16. A double foldable, elastically deformable intraocular lens (IOL) adapted for implanting in the iridocorneal angle of the anterior chamber or ciliary sulcus of the posterior chamber of an eye, said IOL comprising:
a. an elastically deformable optic having an optical axis, a long axis passing through said optical axis and a transverse axis passing through said optical axis orthogonal to said long axis, said optic having a diameter of between about 5 mm and about 7 mm; and
b. first and second flexible fixation members joined to or at opposite edge regions of said optic for fixating said optic in said eye, said first and second fixation members each being generally π-shaped and including respective first and second base regions having lengths parallel to said transverse axis no greater than about 3 mm, and including respective first and second pairs of flexible haptics, proximal ends of said pair of haptics being symmetrically joined to an associated one of said first and second base regions and diverging outwardly from said long axis at an angle between about 30 degrees and about 45 degrees.

17. The double foldable, elastically deformable intraocular lens (IOL) as claimed in claim 16, wherein each one of said pair of flexible haptics is constructed from a material that is stiffer than said optic and has a substantially continuously curved shape and that is more flexible in a proximal end region than in a distal end region and wherein said distal end region is shaped to provide an ocular tissue line contact upon the implanting of said IOL in said eye.

18. The double foldable, elastically deformable intraocular lens (IOL) as claimed in claim 16, wherein distal ends of said pairs of haptics lie on a diameter of between about 11.5 mm and about 13.5 mm that defines the overall diameter of the IOL.

19. A double foldable, elastically deformable intraocular lens (IOL) adapted for implanting in the iridocorneal angle of the anterior chamber or ciliary sulcus of the posterior chamber of an eye, said IOL comprising:
a. an elastically deformable optic having an optical axis, a long axis passing through said optical axis and a transverse axis passing through said optical axis orthogonal to said long axis, said optic having a diameter of between about 5 mm and about 7 mm; and
b. first and second flexible fixation members joined to or at opposite edge regions of said optic for fixating said optic in said eye, said first and second fixation members including respective first and second base regions having lengths parallel to said transverse axis no greater than about 3 mm, said first fixation member being generally π-shaped and including a pair of similar, flexible haptics, proximal ends of said pair of haptics being symmetrically joined to said first fixation member base region and diverging outwardly from said long axis at an angle between about 30 degrees and about 45 degrees, said second fixation member including a single flexible haptic, a proximal ends of said single flexible haptic being joined to said second fixation member base region to extend outwardly from said optic generally along said optic long axis.
